(19) United States
(12) Patent Application Publication
(10) Pub. No.: US 2019/0381214 A1
(43) Pub. Date: Dec. 19, 2019

(54) SCAFFOLD-BASED BRACHYTHERAPY WITH INTEGRATED VISUALIZATION

(71) Applicant: SURGICEYE GMBH, München (DE)

(72) Inventors: Axel BOESE, Magdeburg (DE); Michael FRIEBE, Recklinghausen (DE); Dietmar W. HUTMACHER, Belbowrie (AU)

(73) Assignees: Axel BOESE, Magdeburg (DE); Michael FRIEBE, Recklinghausen (DE); Dietmar W. HUTMACHER, Belbowrie (AU)

(21) Appl. No.: 16/463,860

(22) PCT Filed: Nov. 24, 2017
(86) PCT No.: PCT/EP2017/080294

§ 371 Date: May 24, 2019

(30) Foreign Application Priority Data
Nov. 24, 2016 (DE) 10 2016 122 715.3

(51) Int. Cl. A61L 27/54; A61K 51/12

(52) U.S. Cl. CPC A61L 27/54; A61K 49/0002; A61K 51/1251

(57) ABSTRACT

The invention relates to a material for the 3-dimensional printing of scaffold structures on a 3D printer as well as to a medical implant (1) made of this type of material and the use of this type of material for manufacturing a medical implant (1) of this type. According to the invention, the material has integrated microspheres (5) which emit radioactive rays.

The invention relates to a material for three-dimensional printing of scaffold structures on a 3D printer.

The invention further relates to the field of bio-fabrication, in which the 3D printer is used to produce scaffolds for growing human tissue. This technology can be used inter alia for repairing cartilage, bones, muscles, nerves and skin that have been destroyed by trauma, disease or cancer. For example, implants for breast surgery can be produced, which are used following a breast operation, during resection or partial resection of the breast. Several 100,000 patients are affected by this each year. This technology can also be used for filling surgical cavities in the bones in the case of bone cancer. In the field of tissue filling, the patient's own fat cells are very frequently used today, which cells are then inserted into the surgical cavity. However, the fat can be very quickly absorbed by the patient, and therefore the treatment is very often unsuccessful.

The invention therefore addresses the problem of specifying a material that overcomes the described disadvantages and allows for simple and quick production of scaffold structures.

According to the invention, this problem is solved by a material having the features of claim 1.

The material according to the invention is suitable for three-dimensional printing of scaffold structures on a 3D printer, wherein the material comprises integrated microspheres that emit radioactive rays.

An anti-inflammatory effect and a reduction of rejection reactions can be achieved thereby. Furthermore, local radiation therapy, in particular tumor-bed irradiation, is possible using locally applied, integrated microspheres that emit radioactive rays. This allows for treatment as is known from intravascular brachytherapy and intraoperative therapy. Corresponding implants made of the material can be implanted in the short term, for example for high dose brachytherapy, for a few minutes to hours, or permanently, for example for low dose brachytherapy, having half-lives of up to several years. In this way, rejections by the body can be prevented, and tissue inflammations treated. Furthermore, it is possible to treat cancer cells still present in the tumor bed by this means. Possible printing materials would be for example hydrogels, as starting materials for softer structures, or chitosan or PLA for structures of medium stiffness, and zirconium for hard structures. A type of mini-tripod can be printed using said structural materials. This results in loose bulk material that exhibits a certain spacing between the individual tripods. The microspheres can then be incorporated therein. The tripods are mixed with the microspheres and a carrier fluid or an adhesive fluid to form a suspension, and then applied using a thin applicator. Advantageous embodiments and developments of the invention can be found in the following dependent claims.

According to an advantageous embodiment of the invention, the microspheres are designed as beta and/or gamma emitters. In this case, ideal irradiation is in the order of magnitude of from 3.5 to 14 gray in approximately 2 mm tissue depth. A plurality of beta and gamma emitters are possible for this purpose, in particular 90Y, 1921R, 188Re and 166/167Ho. Yttrium-90 (90Y), for example, as the decay product of strontium-90, is a beta emitter having a half-life of approximately 64 hours that decays, at an average energy of 0.93 megaelectronvolts, to give stable zirconium-90 (90Zr). Yttrium-90 (90Y), for example, is formed as a pellet, preferably encased in glass or resin, having a diameter of 10 μm to 100 μm, and is applied arterially during intravascular and direct tumor treatment of hepatic metastasis. In this case, a pellet has a radioactivity of between 60 and 2000 becquerels. Pellets of this kind can be integrated in the material as microspheres.

In an advantageous embodiment, the microspheres have a half-life of less than a year. A half-life of this kind provides sufficient time for effective treatment using the microspheres.

According to a preferred embodiment, the material is liquid or is provided as a suspension of liquid and solid components. As a result, the material can be very easily worked, applied and made into the desired shape.

A particularly advantageous development is that in which the material hardens in a flexible manner. A material that hardens in a flexible manner is particularly suitable for producing scaffold structures and for filling surgical cavities, since the structure can be adapted to the properties of the target organ of the human body.

A further advantageous embodiment is one in which the material hardens as a porous structure, in particular a spongiform structure. A structure of this kind is particularly suitable as a substitute for endogenous tissue.

According to an advantageous embodiment of the invention, the microspheres comprise a contrast medium that is suitable for magnetic resonance imaging. In this way, the structures formed of the material can be represented with sufficient contrast by means of a non-invasive diagnostic method. As a result, volume rendering of structures formed of the material, in the implanted state, is possible. Furthermore, deformations of the implant formed of the material can be more easily identified on the basis of the non-invasive diagnostic method.

According to an advantageous embodiment of the invention, the microspheres comprise gadolinium. Gadolinium is suitable in particular as a contrast medium positive for magnetic resonance imaging.

An advantageous embodiment is one in which the gadolinium makes up a fraction of from 0.1 ppm to 10% of the mass of the microspheres. This fraction of gadolinium in the microspheres makes it easier to display the structures, formed of the material, by means of magnetic resonance imaging.

A further advantageous embodiment is that in which the microspheres comprise iron oxide as an intensifying contrast medium for magnetic resonance imaging. Iron oxide makes it easier to display the structures, formed of the material, by means of magnetic resonance imaging.

According to a preferred embodiment, the microspheres comprise an X-ray positive contrast medium. In this way, the structures formed of the material can be represented with sufficient contrast by means of the non-invasive diagnostic method. As a result, volume calculations of structures formed of the material, in the implanted state, are possible. Furthermore, deformations of the implant formed of the material can be more easily identified on the basis of the non-invasive diagnostic method.

A particularly advantageous development is that in which the X-ray positive contrast medium makes up a fraction of from 0.1 ppm to 10% of the mass of the microspheres. This fraction of X-ray positive contrast medium in the microspheres makes it easier to display the structures, formed of the material, by means of X-ray imaging.

According to an advantageous embodiment of the invention, the material is biocompatible. A material of this kind does not have any negative influence on the patient who is in direct contact with the implanted structure formed of the material.

According to a preferred embodiment, the material is bioabsorbable. A material of this kind reduces the rejection by the body and tissue inflammation in the case of implanted structures formed of the material. In conjunction with the contrast media described, the absorption by the body of the structure formed of the material can be more easily detected by means of non-invasive diagnostic methods.

The invention furthermore relates to a medical implant, wherein said implant that has already been described and will be described in greater detail in the following, is formed of a material according to the description above and in the following.

The invention furthermore relates to the use of a material according to the description above and in the following for producing a medical implant that has already been described and will be described in greater detail in the following.

Further features, details and advantages of the invention can be found in the following description and in the drawings. Embodiments of the invention are shown purely schematically in the following drawings, and will be described in greater detail in the following. Mutually corresponding objects or elements are provided with the same reference signs in all the figures. In the figures:

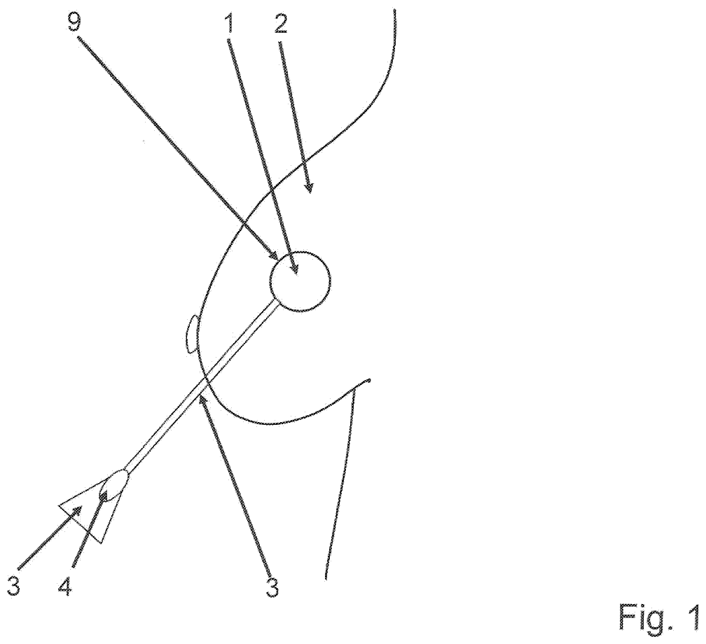

FIG. 1 is a schematic view of a female breast comprising an implant

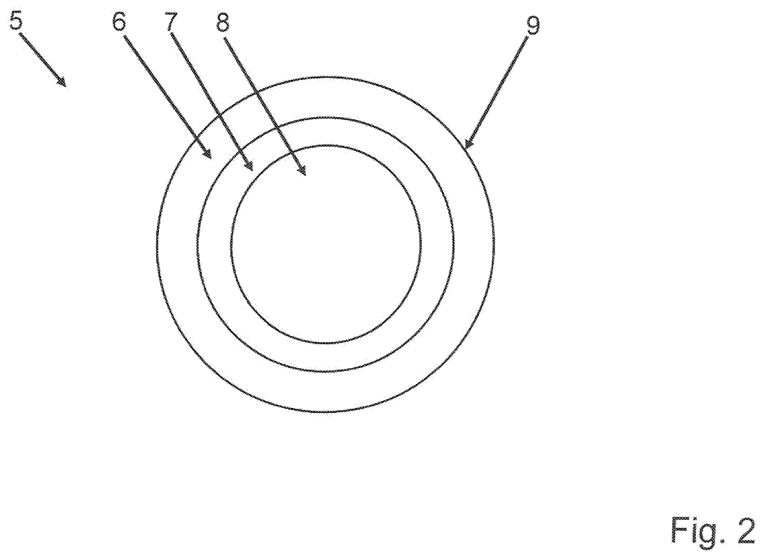

FIG. 2 shows a microsphere

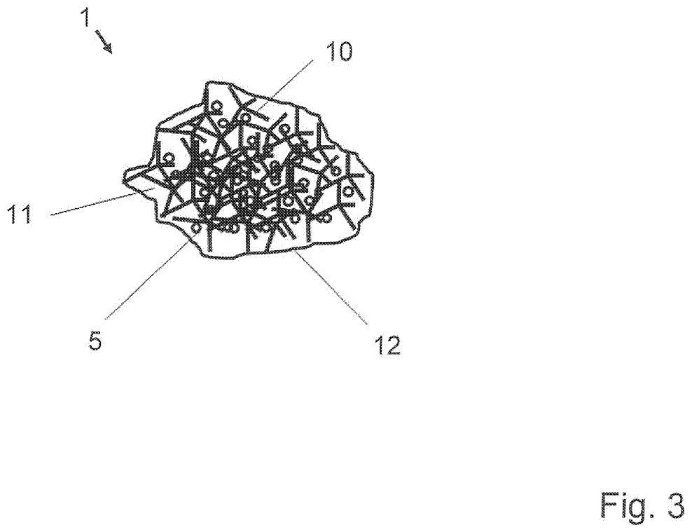

FIG. 3 shows the material

An implant 1, denoted by reference sign 1, is shown schematically in FIG. 1. The drawing according to FIG. 1 furthermore shows a schematic depiction of a female breast 2. The material 4 is introduced in liquid form by means of a catheter 3 or a needle 3, as shown here, and hardens to form an implant 1, shown schematically, in the patient's body. Implants 1 for breast surgery can thus be produced, which are used following a breast operation, during resection or partial resection of the breast 2. The integrated microspheres 5 (FIG. 2) thus allow for local radiation therapy, in particular tumor-bed irradiation, by means of microspheres 5 (FIG. 2) that emit radioactive rays, are integrated in the implant 1, and are used locally in the breast 2. The material can, however, also be printed into a three-dimensional scaffold structure on a 3D printer, which structure, following hardening, is then subsequently implanted during an operation. This is suitable in particular when filling surgical cavities owing to bone cancer. The implant 1, shown schematically, comprises the microspheres 5 (FIG. 2) that are integrated in the material 4, wherein the microspheres 5 (FIG. 2) are designed as beta and/or gamma emitters 6 (FIG. 2). The implant 1 further comprises microspheres 5 (FIG. 2) comprising a contrast medium 7 (FIG. 2) positive for magnetic resonance imaging. Furthermore, the implant 1, which is formed here of the material 4, additionally comprises microspheres 5 (FIG. 2) comprising an X-ray positive contrast medium 8 (FIG. 2). The surface 9 of the implant 1 formed of the material 4 is in addition biocompatible.

FIG. 2 is an enlarged view, by way of example, of an integrated microsphere 5 of the implant 1 (FIG. 1) from FIG. 1. The microsphere 5 preferably has a diameter of approximately 10 μm to 15 μm. As is clearly visible, the beta or gamma emitters 6 on the outer face of the microsphere 5 are located directly on the biocompatible surface 9 of the microsphere 5. Contrast media 7, 8 are arranged on the inside of the microsphere 5, wherein said contrast media are designed so as to be contrast media positive for magnetic resonance imaging or X-ray positive contrast media.

FIG. 3 is a detailed view of the material 4 according to the invention. Mini-tripods, for example, are printed using structural materials 10. Possible structural materials 10 would be for example hydrogels for softer structures, or chitosan or PLA for structures of medium stiffness, and zirconium for hard structures. The structural materials 10 result in loose bulk material that exhibits a certain spacing between the individual tripods. Other geometries are also possible, however. The microspheres 5 can then be incorporated therein. The tripods are mixed with the microspheres 5 and a carrier fluid 11 or an adhesive fluid 11 to form a suspension, and then constitute the material 4 according to the invention. Said material 4 can be applied into the organ cavity 12 using a thin applicator.

Of course, the invention is not limited to the embodiments set out. Further embodiments are possible, without departing from the basic concept.

### LIST OF REFERENCE SIGNS

1 implant
2 female breast
3 catheter or needle
4 material
5 microsphere
6 beta and/or gamma emitter
7 contrast medium positive for magnetic resonance imaging
8 X-ray positive contrast medium
9 surface
10 structural materials
11 carrier fluid or adhesive fluid
12 organ cavity

1. Material (4) for three-dimensional printing of scaffold structures on a 3D printer, wherein the material comprises integrated microspheres (5) which emit radioactive rays.

2. Material (4) according to claim 1, characterized in that the microspheres (5) are designed as beta and/or gamma emitters (6).

3. Material (4) according to claim 1, characterized in that the microspheres (5) have a half-life of less than one year.

4. Material (4) according to claim 1, characterized in that the material (4) is liquid or is a suspension of liquid and solid components.

5. Material (4) according to claim 1, characterized in that the material (4) hardens in a flexible manner.

6. Material (4) according to claim 1, characterized in that the material (4) hardens as a porous structure.

7. Material (4) according to claim 1, characterized in that the microspheres (5) comprise a contrast medium (7) positive for magnetic resonance imaging.

8. Material (4) according to claim 7, characterized in that the microspheres (5) comprise gadolinium.

9. Material (4) according to claim 8, characterized in that the gadolinium makes up a fraction of from 0.1 ppm to 10% of the mass of the microspheres (5).

10. Material (4) according to claim 7, characterized in that the microspheres (5) comprise iron oxide as a contrast medium (7) positive for magnetic resonance imaging.

11. Material (4) according to claim 1, characterized in that the microspheres (5) comprise an X-ray positive contrast medium (8).

12. Material (4) according to claim 10, characterized in that the X-ray positive contrast medium (8) makes up a fraction of from 0.1 ppm to 10% of the mass of the microspheres (5).

13. Material (4) according to claim 1, characterized in that the material (4) is biocompatible.

14. Material (4) according to claim 1, characterized in that the material (4) is bioabsorbable.

15. Medical implant (1), characterized in that it is formed of a material (4) according to claim 1.

16. Use of a material (4) according to claim 1 for producing a medical implant (1).
